(19) United States
(12) Patent Application Publication
(10) Pub. No.: US 2025/0066739 A1
(43) Pub. Date: Feb. 27, 2025

(54) Compositions and Methods for Improving Squamous Epithelial Organoids and Their Production

(71) Applicant: The Trustees of Columbia University in the City of New York, New York, NY (US)

(72) Inventors: Hiroshi NAKAGAWA, Wynnewood, PA (US); Anil K. RUSTGI, New York, NY (US)

(73) Assignee: The Trustees of Columbia University in the City of New York, New York, NY (US)

(21) Appl. No.: 18/946,455

(22) Filed: Nov. 13, 2024

Related U.S. Application Data
(63) Continuation of application No. PCT/US2023/023634, filed on May 26, 2023.
(60) Provisional application No. 63/346,361, filed on May 27, 2022.

(51) Int. Cl. C12N 5/071; C12N 5/09

(52) U.S. Cl. CPC C12N 5/0697; C12N 5/0625; C12N 5/0693; C12N 2501/11; C12N 2501/119; C12N 2503/04; C12N 2513/00; C12N 2533/90

(57) ABSTRACT

Media for making esophageal organoids, methods of making an esophageal organoid using that media, and esophageal organoids produced by those methods

### CROSS-REFERENCE TO RELATED APPLICATIONS

This is a continuation of International Patent Application No. PCT/US2023/023634 filed on 26 May 2023, which claims priority to U.S. Provisional Patent Application No. 63/346,361 filed on 27 May 2022, each of which are incorporated herein in their entirety.

### STATEMENT REGARDING FEDERALLY SPONSORED RESEARCH OR DEVELOPMENT

This invention was made with government support through grants R01AA026297, PO1CA098101, R01DK114436, and U54CA163004 awarded by the National Institutes of Health. The government has certain rights in the invention.

### TECHNICAL FIELD OF THE INVENTION

This disclosure generally relates to the fields of tissue engineering and organ modeling. More specifically, this disclosure relates to three-dimensional (3D) squamous epithelial organoids that recapitulate the morphology and function of original squamous epithelia of various organs including the head-and-neck (mouth and throat), the esophagus, the uterus, as well as related compositions and methods.

### BACKGROUND OF THE INVENTION

Esophageal cancers comprise esophageal adenocarcinoma (EAC) and esophageal squamous cell carcinoma (ESCC), two distinct histologic subtypes. Both EAC and ESCC are amongst the deadliest of all human malignancies featuring presentation at late stages, therapy resistance, early recurrence, and poor prognosis. Grown rapidly ex vivo, patient-derived organoids (PDO) recapitulate the original tissue architecture of primary esophageal tumors.

Protocols for generating and characterizing esophageal cancer PDO growth, morphology, and biology are known. For example, tissue specimens (diagnostic biopsies or surgically resected tumor tissues) are subjected to enzymatic and mechanical disruption in order to obtain single-cell suspensions, which are embedded in basement membrane matrix (MATRIGEL®) and cultured in unique organoid growth media optimized for distinct histologic tumor types (i.e., adenocarcinoma vs. squamous cell carcinoma). Following two weeks of culturing, the resulting primary PDO are passaged, cryopreserved, or harvested for morphological and functional analyses.

The harvested organoids can be subjected to a variety of morphological and functional assays including, but not limited to, immunohistochemistry, immunofluorescence, Western blotting, flow cytometry, quantitative polymerase chain reaction and RNA-sequencing (bulk and single-cell). The conditioned media from organoid cultures can be used for enzyme-linked immunosorbent assays. Passaged organoids can be tested for conventional and experimental therapeutics in a moderate-to-high throughput manner. Drug treatment of 3D organoids with variable concentrations of therapeutic agents determines their half maximal inhibitory concentration (IC50). Analysis of surviving cells provides insights into the potential drug resistance mechanisms. Taken together, current protocols provide a comprehensive experimental platform to study the molecular mechanisms underlying esophageal cancer cell propagation and drug responses.

However, these same protocols are not without their shortcomings. For example, the success rate of producing PDOs that can be successfully passaged a significant number of times (e.g., ten or more) is only ˜60%. Accordingly, modifications to existing protocols are required in order to increase the current success rate.

### BRIEF SUMMARY OF THE INVENTION

The present disclosure relates to organoid media for producing an esophageal organoid, as well as methods of using that media to produce esophageal organoids, and esophageal organoids produced using those methods.

In some embodiments, the inventions disclosed herein relate to an organoid medium for producing an esophageal organoid, comprising: (a) a solubilized membrane matrix (e.g., MATRIGEL®); (b) about 1 μM to about 20 μM of a TGF-B inhibitor (e.g., A83-01, SB-431542); and (c) less than about 50 ng/ml of epidermal growth factor (EGF). In some embodiments, the medium further comprises from about 0.5×to about 1.5×N-2 supplement. In some embodiments, the medium further comprises advanced DMEM+/+/+, 1×B27 supplement, 1.25 mmol/L N-acetyl-L-cysteine, 4% R-spondin, and 4% Noggin. In some embodiments, the medium comprises a feeder layer comprising a plurality of cancer associated fibroblasts or fetal esophageal fibroblasts.

In some embodiments, the inventions disclosed herein relate to methods for producing an esophageal organoid, comprising: (a) isolating cells from an esophageal biopsy to provide isolated cells; (b) culturing the isolated cells in a three-dimensional culture comprising an organoid medium for a time sufficient to produce at least one organoid, wherein the organoid medium comprises (i) a solubilized membrane matrix; (ii) about 1 μM to about 20 M of a TGF-B inhibitor; and (iii) less than about 50 ng/ml of epidermal growth factor (EGF). In some embodiments, the medium further comprises from about 0.5×to about 1.5×N-2 supplement. In some embodiments, the medium further comprises advanced DMEM+/+/+, 1×B27 supplement, 1.25 mmol/L N-acetyl-L-cysteine, 4% R-spondin, and 4% Noggin. In some embodiments, the medium comprises a feeder layer comprising a plurality of cancer associated fibroblasts or fetal esophageal fibroblasts. In some embodiments, the cells are cells from an esophageal adenocarcinoma (EAC) or esophageal squamous cell carcinoma (ESCC).

In some embodiments, the inventions disclosed herein relate to an esophageal organoid produced by any of the foregoing methods. In some embodiments, the esophageal organoid is produced from cells from an esophageal adenocarcinoma (EAC) or esophageal squamous cell carcinoma (ESCC). In some embodiments, the esophageal organoid which is capable of being sub-cultured for at least 10 passages.

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below in the Detailed Description of the Invention. This summary is not intended to identify key features or essential features of the claimed subject matter, nor is it intended to be used as an aid in determining the scope of the claimed subject matter.

### BRIEF DESCRIPTION OF THE SEVERAL VIEWS OF THE DRAWINGS

FIG. 1 shows that EGF concentration influences organoid formation and structure in esophageal 3D organoids. EPC1 and EPC2 are hTERT-immortalized normal esophageal keratinocytes.

FIG. 2 shows that EGF may stimulate growth, if not formation, of neoplastic PDOs.

FIG. 3 shows organoid formation rate in established PDOs (P5 or later) when grown in media in accordance with the inventions disclosed herein.

FIG. 4 shows organoid size in established PDOs (P5 or later) when grown in media in accordance with the inventions disclosed herein.

FIG. 5 shows that inhibition of TGF-β receptor signaling improves organoid formation by normal epithelial cells. EPC1 and EPC2 are hTERT-immortalized normal esophageal keratinocytes. EPC1/EPC2 organoids did not grow in HOME50 devoid of A83-01 (ΔA).

FIG. 6 shows that inhibition of TGF-β receptor signaling permits PDO formation by a subset of SCC tumor samples.

FIG. 7 shows the dose-dependent effects of TGF-β receptor signaling inhibitors upon organoid formation by ESCC1 and OCTT102 cells.

FIG. 8 shows that inhibition of TGFβ receptor signaling may extend replicative lifespan in a subset of neoplastic PODs

FIGS. 9-13 show organoid formation rate (OFR) and population doubling (PDL) measured at various time points for organoids grown in different media (KSFMC, HOME0, HOME50, or HC). Organoids EN2, HN1*, HN11 were grown from normal mucosa (EN, esophageal normal; HN, head-and-neck normal). HSC5 and HSC6 were from head and neck squamous cell cancer tumors. HSD5 and HSD6 were from head and neck/oral prencoplasia/dysplasia.

FIG. 14 shows that PDO was established from a 78 y.o. male with oral (tongue) squamous dysplasia. Established organoids were grown indefinitely (>10 passages) in HOME50 medium, displaying moderate atypia (grade 2). Scale bar, 100 μm.

FIGS. 15 and 16 show organoid formation rate (OFR) and population doubling (PDL) measured at various time points for organoids grown in different media (KSFMC, HOME0, HOME50, or HC). Organoids EN2, HN1*, HN11 were grown from normal mucosa (EN, esophageal normal; HN, head-and-neck normal). HSC5 and HSC6 were from head and neck squamous cell cancer tumors. HSD5 and HSD6 were from head and neck/oral preneoplasia/dysplasia.

FIG. 17 shows the effect of co-culturing PDOs in the presence of a feeder layer of cancer associated fibroblasts (CAFs). After 48 hours there is a clear difference in the number of organoids after two days, which becomes even more pronounced by day four and is still significant at day five.

### DETAILED DESCRIPTION OF THE INVENTION

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

Unless otherwise defined herein, scientific, and technical terms used in connection with the present invention shall have the meanings that are commonly understood by those of ordinary skill in the art. Further, unless otherwise required by context, singular terms shall include pluralities and plural terms shall include the singular. More specifically, as used in this specification and the appended claims, the singular forms “a,” “an” and “the” include plural referents unless the context clearly dictates otherwise. Thus, for example, reference to “a protein” includes a plurality of proteins; reference to “a cell” includes mixtures of cells, and the like.

In addition, ranges provided in the specification and appended claims include both end points and all points between the end points. Therefore, a range of 1.0 to 2.0 includes 1.0, 2.0, and all points between 1.0 and 2.0.

The term “about” as used herein when referring to a measurable value such as an amount, a temporal duration, and the like, is meant to encompass variations of .+−.20%, .+−.10%, .+−.5%, .+−.1%, or .+−.0.1% from the specified value, as such variations are appropriate to perform the disclosed methods.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of “consisting essentially of,” when used in the claims, shall have its ordinary meaning as used in the field of patent law.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of and “consisting essentially of shall be closed or semi-closed transitional phrases, respectively.

Generally, nomenclature used in connection with, and techniques of, cell and tissue culture, molecular biology, immunology, microbiology, genetics and protein and nucleic acid chemistry and hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art.

The inventions described herein relate to media for making esophageal organoids, methods of making an esophageal organoid using that media, and esophageal organoids produced by those methods as described hereinbelow.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Generation of Esophageal Cancer PDO

A tissue specimen is obtained via diagnostic biopsy or surgery (esophagectomy or endoscopic mucosal resection) and dissociated by enzymatic digestion (Dispase and Trypsin), and embedded into a single-cell suspension in MATRIGEL® matrix. PDO are grown in tumor type-specific organoid medium at 37° C. under a controlled atmosphere with 5% CO2 and 95% relative humidity, resulting in formation of spherical 3D structures representative of the original tumor.

### Example 2

### Dissociation of Human Esophageal Biopsies to a Single Cell Suspension

Tumor tissue was transferred to with sterile forceps into a 60-mm cell culture dish with sterile forceps, and minced into smaller fragments (<1 mm) with sterile dissecting scissors. Minced tissue fragments were subsequently transferred into a 1.7-mL tube containing 1 mL HBSS-DF. The mixture was incubated at for 10 min at 37° C. with simultaneous mixing at 800 rpm in Thermomixer C. Collagenase IV and Y-27632 may optionally be added into HBSS-DF (HBSS-DFCY) with an extended incubation time period for ˜45 min to increase the yield of single cells.

The mixture was subsequently centrifuged for ˜10 sec at room temperature. The supernatant was removed using a single-channel P1000 pipettor with a 1,250-μL tip, and the pellet re-suspended with 1 mL 0.25% trypsin-EDTA and incubated for 10 min at 37° C. with simultaneous mixing at 800 rpm in a Thermomixer C. DNase I may optionally be added to the re-suspended pellet (0.5 U/mL) in order to degrade DNA released from broken cells to minimize cell aggregates.

Trypsinized tissue fragments were filtered over a 100-μm strainer into a 50-mL tube containing 8 mL STI. ˜8 mL of the filtrate (cell suspension) was removed from the 50-mL tube using a 10-mL pipette and used to wash the strainer three times. The filtrate was transferred into a 15-mL tube and centrifuged at 188×g (1,000 rpm on Sorvall ST 16R) for 5 min at 4° C. The supernatant was subsequently discarded, the cell pellet was resuspended in 1 mL Basal Medium. Cell density and cell viability (via Trypan Blue exclusion test) were determined using a COUNTESS™ II Automated Cell Counter.

### Example 3

PDO Initiation and Growth in 24-well Plates

In general, 2×10⁴ cells were seeded per well to initiate PDO in 24-well plates, which were pre-warmed to 37° C. 1.4×10⁵ cells were transferred into a 1.7-mL tube and centrifuged at 500×g (2,300 rpm on Eppendorf 5424R) for 3 min at room temperature to pellet cells. Cells were resuspended in 350 μL ice-cold MATRIGEL® and 50 μL of the suspension was aliquoted into each well of 24-well plate. The 24-well plate was placed in a CO₂ incubator at 37° C. for 30 min to allow the MATRIGEL® to solidify.

500 μL of a tumor-type specific organoid medium (see Example 11) was added to each well, and the organoid media was changed every 2 to 3 days. Contamination and organoid growth was monitored under a phase-contrast microscope, and organoids were allowed to grow for 10 to 14 days and then passaged or harvested. If spherical structures were observed but grew slowly, the culturing period was extended for another 7 to 14 days.

### Example 4

### Propagation and Cryopreservation of Esophageal Cancer PDO

Once established, esophageal cancer cells were isolated from the primary PDO by enzymatic dissociation to seed subsequent passages (i.e., sub-culture) in order to propagate further for histological analyses and flow cytometry as described in subsequent Examples. PDO was also sub-cultured in 96-well plates for drug treatment experiments described in yet another Example. Additionally, isolated esophageal cancer cells were cryopreserved for long-term storage.

Culture media was carefully removed using a pipette so as not to disturb the MATRIGEL® containing growing mature organoids in each well. 500 μL of cold DPBS was added into each well and MATRIGEL® was mechanically disrupted into small fragments using a pipette. PDO-containing MATRIGEL® from every 3 wells was combined, transferred into a 1.7-mL tube, and centrifuged at 500×g (2,300 rpm on Eppendorf 5424R) for 3 min at room temperature to pellet the fragments. Supernatant was discarded, and the fragments were re-suspended in 1 mL Trypsin-EDTA optionally supplemented with DNase I as described above.

The cell suspension was incubated for 10 min at 37° C. with simultaneous mixing at 800 rpm in Thermomixer C. To disintegrate organoid structures further, the cell suspension was pipetted 3 to 4 times out of and back into the tube.

The cell suspension was strained over a 35-μm cell strainer cap into a 5-mL Falcon round-bottom tube containing 3 mL STI and subsequently centrifuged at 188×g (1,000 rpm on Sorvall ST 16R) for 5 min at 4° C. The supernatant was aspirated and the cell pellet was resuspended in 1 mL of Basal Medium. Cell density and viability were determined as descried above.

### Example 5

### Passaging PDO

In order to prepare 96-well plates for drug treatment experiments, a total of 6×10⁴ cells (per plate) were initially transferred into a 1.7-mL tube and centrifuged at 500×g (2,300 rpm on Eppendorf 5424R) at room temperature for 3 min to pellet cells. Cells were subsequently re-suspended in ice-cold MATRIGEL®, and 5 μL of the cells-in-MATRIGEL® suspension was dispensed into each well. Each 96-well plate was placed in a CO2 incubator (37° C.) for 30 min to allow the MATRIGEL® to solidify. 100 μL of tumor-type specific organoid media was then dispensed into each well for ESCC or EAC according to clinical diagnosis and pathology report of the original tumor. Organoid media was refreshed every 2 to 3 days while monitoring for contamination and organoid growth under a phase-contrast microscope as described above. Organoids were allowed to grow until reaching 70-100 μm in diameter, which typically took about 7 to 8 days.

### Example 6

### Cryopreservation and Recovery

To make a vial of frozen stock, 1×10⁵ live cells were transferred into a 1.7-mL tube and centrifuged at 500×g (2,300 rpm on Eppendorf 5424R) at room temperature for 3 min to pellet cells. After removing the supernatant, the cells were resuspended in 1 mL of freezing media to achieve a final cell density of 3×10⁵ cells/mL. The suspension was aliquoted into three fresh cryogenic vials (330 μL), to which 670 μL of freezing medium was added. The vials were stored overnight at −80° C. in a freezing container and then transferred to a liquid nitrogen cell storage tank.

Once needed for use, cryogenic vials were thawed in a 37° C.—water-bath for 30-45 sec and the cell suspension was transferred to a 1.7-mL tube and centrifuged at 500×g (2,300 rpm on Eppendorf 5424R) at room temperature for 3 min to pellet cells. The cell pellet was resuspended in 1 mL DPBS and centrifuged again at 500×g (2,300 rpm on Eppendorf 5424R) at room temperature for 3 min to pellet cells. The cell pellet was resuspended in 1 mL Basal Medium, so that cell density and viability could be determined. Once determined, the cell suspension was again centrifuged at 500×g (2,300 rpm on Eppendorf 5424R) at room temperature for 3 min to pellet cells, and the pellet was resuspended in MATRIGEL® in order to proceed with organoid culture as described previously. For EAC PDO, it was discovered that supplementation with 100 ng/ml FGF-10 in the first week dramatically increased recovery after cryopreservation.

### Example 7

### Imaged-based Monitoring of Organoid Size and Growth Kinetics

To evaluate organoid growth, 3D organoid structures were monitored during culturing, and their size was documented by a conventional phase-contrast inverted microscope or high-throughput cell imaging instruments (e.g., Celigo Image Cytometer, Nexcelom Bioscience) with the capacity of automated multi-well rapid imaging and quantitative analysis of organoid size, number, and structure. Organoids were grown in 24-well or 96-well plates as described above, and phase-contrast images were manually acquired. Using ImageJ software, the diameter of at least 5 organoids per well was measured. The Celigo Image Cytometer was also used to acquire phase-contrast images and calculate mean organoid area for all organoids imaged. Growth curves were subsequently calculated by repeating measurements every other day and plotting organoid diameter or area at each time point.

### Example 8

### Harvesting Esophageal Cancer PDO for Histological Analyses

Histological evaluation is an essential step in ensuring that PDO recapitulate the original tumor morphologically. Hereinbelow is provided a protocol for fixation and embedding of PDO in paraffin, allowing for a long-term storage and histological analyses including hematoxylin-eosin staining, immunohistochemistry, and immunofluorescence.

Initially, culture media was removed from each well and replaced with 500 μL of cold DPBS. MATRIGEL® was dislodged and disrupted as described above, and fragmented MATRIGEL® from three wells was combined and transferred into a 1.7-mL tube. The cell pellet was centrifuged at 500×g (2,300 rpm on Eppendorf 5424R) at room temperature for 3 min to pellet MATRIGEL® fragments. After removing the supernatant, 1 mL DPBS was added to the pellet for further dissociation using a pipette. The cell pellet was centrifuged again at 500×g (2,300 rpm on Eppendorf 5424R) at room temperature for 3 min to pellet MATRIGEL® fragments. After removing the supernatant, the pellet was resuspended in 500 μL 4% PFA and incubated at 4° C. for at least 2 hours.

The suspension was then centrifuged again at 500×g (2,300 rpm on Eppendorf 5424R) at room temperature for 3 min. After removing the supernatant, 1 mL DPBS was added to the pellet to wash the pellet by pipette. The suspension was again centrifuged and supernatant was removed.

Cells were subsequently embedded in paraffin by placing embedding gel (5 ml in a 15-mL tube) in a 150-mL beaker containing ˜100 mL water, and then microwaving the beaker until the water began to boil. The resultant liquified embedding gel was then allowed to sit for 2 to 3 minutes, and then the organoid pellet was resuspended in 50 μL of liquefied embedding gel using an embedding tip on P200 pipettor. The resuspended pellet was immediately transferred and cast into an embedding bottom-less barrel placed on a parafilm-covered embedding rack. The embedding rack was subsequently transferred to a refrigerator at 4° C. in order to let the gel solidify (>30 min). After solidifying, the gel was transferred to sponge placed within a tissue cassette. The tissue cassette was placed into 70% ethanol and stored at 4° C. until embedding in paraffin via routine histological processing to prepare paraffin blocks.

### Example 9

### PDO Content Analysis by Flow Cytometry

Single cell-derived PDO recapitulate intratumoral cell heterogeneity. Besides morphology, PDO content can be characterized using flow cytometry to measure, e.g., cell surface markers. Such analysis can be done in conjunction with pharmacological treatments to explore unique signaling pathways or therapy resistance mechanisms associated with unique cell populations within PDO. Fluorescence-labeled antibodies, dyes and probes can be utilized to detect a variety of cellular antigens and molecular targets.

Initially, cell suspensions were created from organoids as previously described and cell density was determined as described above. 2×10⁵-1×10⁶ cells were transferred to a 5-mL Falcon round-bottom tube. 4 mL of FACS buffer was added to the tube and then the tube was centrifuged at 188×g (1,000 rpm on Sorvall ST 16R) at 4° C. for 5 min. Supernatant was removed and 100 μL of FACS buffer containing 5 μL of a conjugated antibody (e.g., anti-CD44 antibody) in cell suspension (1:20, pre-optimized titer) was added to the tube. The contents of the tube were vortexed and then the contents of the tube were incubated on ice and in the dark for a time sufficient to optimize antibody binding (e.g., 30 min).

4 mL of FACS buffer was subsequently added to the tube, and cells were washed by centrifugation at 188×g (1,000 rpm on Sorvall ST 16R) at 4° C. for 5 min. The supernatant was discarded and cells were resuspended in 500 μL FACS buffer containing 1 μL DAPI. DAPI-negative cells were analyzed for antigen (e.g., CD44) expression using a flow cytometer and analysis software.

### Example 10

### Evaluation of Drug Response by Determination of the Half-inhibitory Concentration (IC₅₀)

One of the major goals in PDO translation is to serve as a potential guide to assist clinical decision-making by physicians and surgeons in personalized/precision medicine where customized therapeutics are provided following molecular characterization of cancer cells in the original tumors. To this end, PDO need to be tested for multiple drugs in standard of care and molecularly-targeted agents (e.g., small molecule inhibitors and antibodies) in a time-sensitive manner. Drug treatment of PDO can be performed in 96-well plates containing established PDO with a broad range of drug concentrations. PDO response to drugs can be evaluated via numerous cell viability assays based upon cellular functions (e.g., ATP production and other mitochondrial activities such as formazan formation in the WST-1 reagent) and cell membrane integrity (e.g., membrane-permeating fluorescent dyes such as Calcein-AM).

In order to carry out drug experiments, culture media was first removed from all organoid-containing wells. 100 μL of organoid medium containing drugs at a desired concentration or range of concentrations was then dispersed into each well and allowed to incubate with organoids for 72 hours.

In order to determine cell viability, drug-containing media was removed from each well replaced with 100 μL volume of a 1:1 cocktail of CELLTITER-GLO® 3D reagent and Basal Medium. After determining background luminescence according to protocol, the contents of each well were vigorously mixed for 4 for 5 min to induce cell lysis. Plates were incubated at room temperature for 25 min and luminescence was measured using a GloMax-Multi+ Microplate Multimode Reader. Dose response curves were then generated using any number of methods known in the art.

### Example 11

### Preparation of Basal Medium and Tumor Type-specific Organoid Media

TABLE 1
Basal Media

| | | |
|---|---|---|
| Reagent | Volume | Final concentration |

| Advanced DMEM/F12 | 500 | mL | | |
|---|---|---|---|---|
| GlutaMAX (100x) | 5 | mL | 1x | |
| HEPES (1M) (pH 7.2-7.5) | 5 | mL | 10 | mM |
| Antibiotic-Antimycotic (100x) | 5 | mL | 1x | |
| Gentamicin (50 mg/mL) | 50 | μL | 5 | μg/mL |

TABLE 2
ESCC Organoid Medium (50 mL)

| | | |
|---|---|---|
| Reagent | Volume | Final concentration |

| Basal Medium | 47 | mL | | |
|---|---|---|---|---|
| RN conditioned medium | 1 | mL | 2% | |
| N-2 (100x) | 500 | μL | 1x | |
| B-27 (50x) | 1 | mL | 1x | |
| NAC (0.5M) | 100 | μL | 1 | mM |
| EGF (500 ng/μL) | 5 | μL | 50 | ng/mL |
| Y-27632 (10 mM) * | 50 | μL | 10 | μM ** |
| Gentamicin (50 mg/mL) | 5 | μL | 10 | μg/mL |

| | | | |
|---|---|---|---|
| Antibiotic-Antimycotic (x100) | 500 | μL | 1x |

* only needed when establishing during day 0-day 2
** Basal Medium contains 5 μM Gentamicin before this supplementation.

TABLE 3
EAC Organoid Medium (50 mL)

| | | |
|---|---|---|
| Reagent | Volume | Final concentration |

| Basal Medium | 24 | mL | | |
|---|---|---|---|---|
| WRN Conditioned Medium | 24 | mL | 50% | |
| N-2 (100x) | 500 | μL | 1x | |
| B-27 (50x) | 1 | mL | 1x | |
| NAC (0.5M) | 100 | μL | 1 | mM |
| CHIR99021 (5 mM) | 5 | μL | 0.5 | μM |
| EGF (500 ng/μL) | 25 | μL | 250 | ng/mL |
| A83-01 (5 mM) | 5 | μL | 0.5 | μM |
| SB202190 (10 mM) | 5 | μL | 1 | μM |
| Gastrin (1 mM) | 5 | μL | 0.1 | μM |
| Nicotinamide (1M) | 1 | mL | 20 | mM |
| Y-27632 (10 mM) | 50 | μL | 10 | μM |
| Gentamicin (50 mg/mL) | 5 | μL | 10 | μM * |
| Antibiotic-Antimycotic (x100) | 500 | μL | 1x | |
| FGF-10 (100 μg/mL) ** | 50 | μL | | |

* Basal Medium contains 5 μM Gentamicin before this supplementation.
** only added when establishing primary cultures and recovering from frozen stocks.

TABLE 4
HEK293T medium

| Reagent | Volume | | Final concentration |
|---|---|---|---|
| DMEM | 500 | mL | |
| FBS | 50 | mL | 10% |
| Penicillin-Streptomycin (100x) | 5 | mL | 1x |

Various reagents were combined to generate basal as well as tumor-type specific organoid media as described in Tables 1-4 above.
Advanced DMEM/F12 (Thermo Fisher Scientific, cat. No. 12634028)
GlutaMAX supplement, 100× (Thermo Fisher Scientific, cat. No. 35050061).
HEPES, 1 M (pH 7.2-7.5) (Thermo Fisher Scientific, cat. No. 15630080).
Antibiotic-Antimycotic, 100× (Thermo Fisher Scientific, cat. No. 15240062)
Gentamicin, 50 mg/mL (Thermo Fisher Scientific, cat. No. 15750060)
N-2 Supplement, 100× (Thermo Fisher Scientific, cat. No. 17502048).
B-27 supplement, 50× (Thermo Fisher Scientific, cat. No. 17504044)
N-Acetylcysteine (NAC), 0.5M (Sigma-Aldrich, cat. No. A9165), reconstituted in DPBS, filter-sterilized and stored in aliquots at −20° C.
CHIR99021, 5 mM (Cayman Chemical, cat. No. 13122), reconstituted in DMSO, stored in aliquots at −20° C.
Recombinant human epidermal growth factor (EGF), 500 ng/μL (Peprotech, cat. No. AF-100-15), reconstituted in Basal Medium, stored in aliquots at −20° C.
A83-01, 5 mM (Cayman Chemical, cat. No. 9001799), reconstituted in DMSO, stored in aliquots at −20° C.
SB202190, 10 mM (Selleck Chemicals, cat. No. S1077), reconstituted in DMSO, stored in aliquots at −20° C.
Gastrin, 1 mM (Sigma-Aldrich, cat. No. G9145), reconstituted in sterile 0.1% NaOH, stored in aliquots at −20° C.
Nicotinamide, 1M (Sigma-Aldrich, cat. No. N0636), reconstituted in DPBS, filter-sterilized and stored in aliquots at −20° C.
Y-27632, 50 mM (Selleck Chemicals, cat. No. S1049), reconstituted in DMSO, stored in aliquots at −20° C.
FGF-10, 100 μg/mL (Peprotech, Cat. No. 100-26), reconstituted in Basal medium, stored in aliquots at −20° C.
L-WRN cell-conditioned medium expressing Wnt-3A, R-Spondin1 and Noggin (WRN), stored at −20° C.
L-WRN cells (ATCC Cat. No. CRL-3276).
HEK293T-conditioned medium expressing R-Spondin1 and Noggin (RN), stored at −20° C.

### Example 12

### Conditioned Medium Containing High RN Activity

To generate conditioned medium containing high RN activity, high-titer lentivirus expressing RN was produced by transient transfection of HEK293T cells. The resulting high-titer virus-containing HEK293T cell conditioned medium was used to infect HEK293T cells to produce RN that was harvested as a conditioned medium from virus-infected HEK293T cells.

Organoid culture media require developmental niche factors (i.e., WNR or NR). Such factors can be harvested as cell culture conditioned media, providing a more affordable alternative to commercially available recombinant proteins. Provided hereinbelow is a protocol to produce RN in HEK293T cells via lentivirus-mediated transduction of R-spondin1 and Noggin. The produced RN can be validated in murine small-intestinal organoid formation assays).

HEK293T cells were initially grown in in HEK 293T medium at 5% CO₂ and at 37° C. under >95% humidity. The cells were washed with DPBS, trypsinized, and counted using standard cell culture procedures. 6×10⁶ HEK293T cells were seeded in a 100 mm dish and grown for 48-72 h to 80-90% confluency in the following day.

40 μL of lipofectamine 2000 was mixed into 360 μL of Opti-MEM into a first tube and incubated at room temperature for 5 min. DNA (10 μg pG−N+RIP, 6.5 μg pCMVdR8.74, 3.5 μg VSV.G) was added to 400 μL of Opti-MEM in a second tube. The contents of the first and second tube were combined and incubated at room temperature for 30 min. 5 mL of Opti-MEM was added to the Opti-MEM-lipofectamine-DNA cocktail.

Media was removed from the HEK293T cell culture and replaced with the Opti-MEM-lipofectamine-DNA cocktail and allowed to incubate at 37° C. for 4 h. Media was removed and replaced with 7 mL of HEK293T medium. Cells were incubated at 5% CO₂ and at 37° C. under >95% humidity for 48-72 h. Virus was harvested at 48 and 72 h as conditioned medium and filtered using a 0.45-μm syringe filter.

HEK293T cells were subsequently plated into a 100 mm dish and allowed to grow to 80-90% confluency. A control plate using puromycin (final concentration of 2 μg/mL) was also run. Culture medium was replaced with 7 mL virus-conditioned medium supplemented with Polybrene (3.5 μL of 10 mg/mL stock) and incubated at 37° C. for 4 h. 3 mL of HEK293T medium was subsequently added to the dish and and incubates at 37° C. for 4 h. Virus-conditioned medium was replaced and incubated for 24 hours.

Cells were washed with DPBS and trypsinized. 5×10⁶ cells were seeded into as many 150 mm dishes as possible in HEK293T medium without puromycin and media was collected at 24, 48 and 72 hours and stored at −80° C. Conditioned media was combined and filter sterilized as described above and stored at −80° C.

### Example 13

### Optimization of PDO Culture Conditions

In order to improve the success rate of creating and subculturing organoids through increasing numbers of passages, alterations to existing media were explored and compared to each other and the HC media described in Driehuis et al., Cancer Discov, 2019; 9:852-71. Briefly, the HC media is comprised of advanced DMEM+/+/+ (DMEM/F-12 containing GLUTAMAX™ and HEPES supplemented with antibiotics), 1×B27 supplement (Life Technologies, catalog no. 17504-044), 1.25 mmol/L N-acetyl-L-cysteine (Sigma-Aldrich, catalog no. A9165), 10 mmol/L nicotinamide (Sigma-Aldrich, catalog no. N0636), 50 ng/mL human EGF (PeproTech, catalog no. AF-100-15), 500 nmol/L TGF-β inhibitor A83-01, 10 ng/mL human FGF10 (PeproTech, catalog no. 100-26), 5 ng/mL human FGF2 (PeproTech, catalog no. 100-18B), 1 μmol/L Prostaglandin E2 (Tocris Bioscience, catalog no. 2296), 0.3 μmol/L CHIR 99021 (Sigma-Aldrich, catalog no. SML1046), 1 μmol/L Forskolin [Bio-Techne (R&D Systems) catalog no. 1099], and 4% R-spondin, and 4% Noggin.

The new media were given internal reference identifiers HOME, HOME5 and KSFMC, HOME0 and HOME50 are comprised of advanced DMEM+/+/+, B27 supplement, N-acetylcysteine (NAC), Noggin and R-spondin1, but do not contain FGF2, FGF10, CHIR99021, Forskolin, and prostaglandin E2 used in HC medium. HOME0 also lacks EGF. HOME0 and HOME50 contain N2 supplement that is not used in HC. KSFMC is a modified version of keratinocyte serum-free medium (KSFM; Invitrogen/Thermo Fisher Scientific) with 0.6 mM CaCl₂ and 1 ng/mL EGF. KSFM does not facilitate organoid growth.

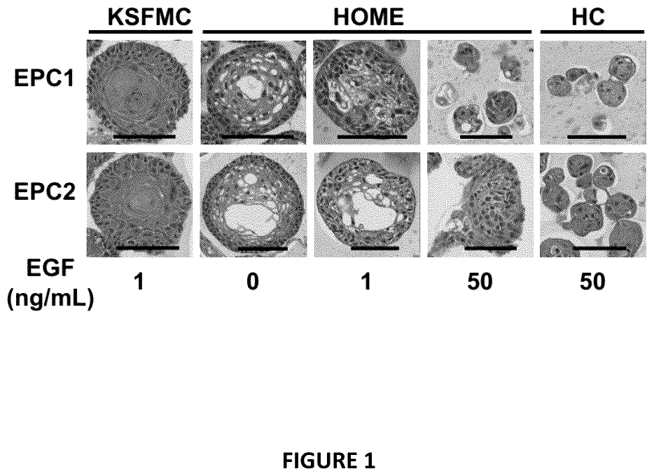

As shown in FIG. 1, EGF concentration influences organoid formation and structure in esophageal 3D organoids. Low EGF concentration more closely recapitulates a normal squamous-cell differentiation gradient.

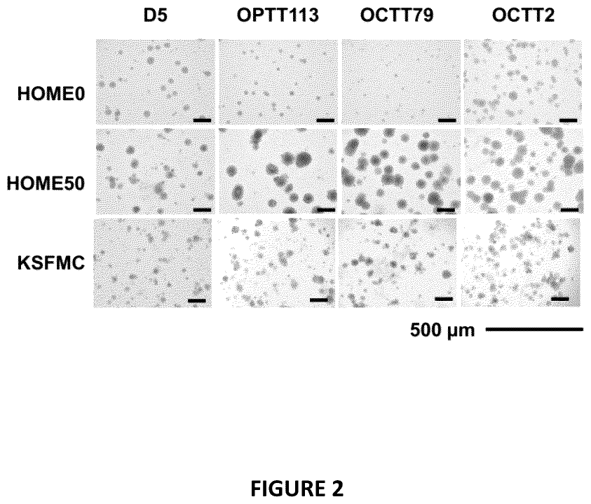

As shown in FIG. 2, EGF may stimulate growth, if not formation, of neoplastic PDOs.

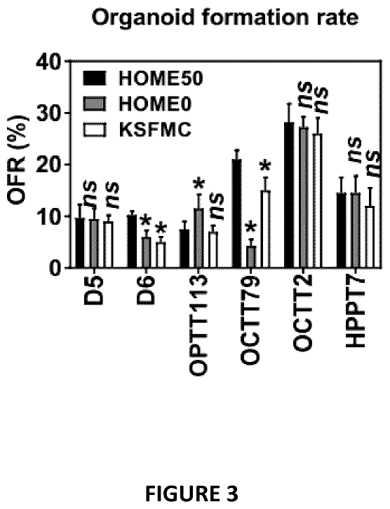

FIG. 3 shows organoid formation rate (OFR) in established PDOs (P5 or later) when grown in HOME50, HOME0 and KSFMC media.

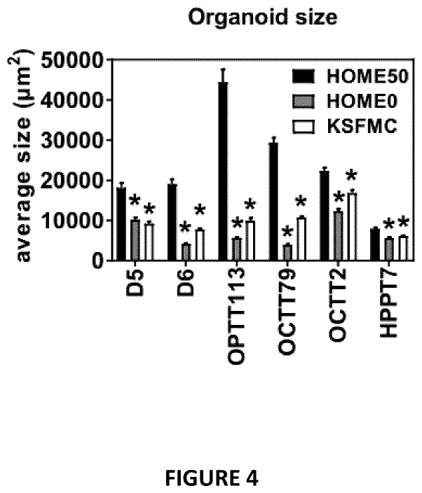

FIG. 4 shows organoid size in established PDOs (P5 or later) when grown in HOME50, HOME0 and KSFMC media.

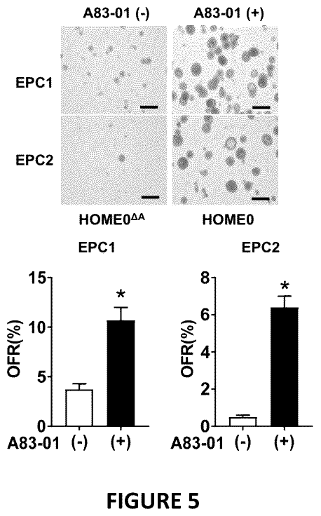

FIG. 5 shows that inhibition of TGF-β receptor signaling improves organoid formation by normal epithelial cells. EPC1 and EPC2 are hTERT-immortalized normal esophageal keratinocytes. EPC1/EPC2 organoids did not grow in HOME50 devoid of A83-01.

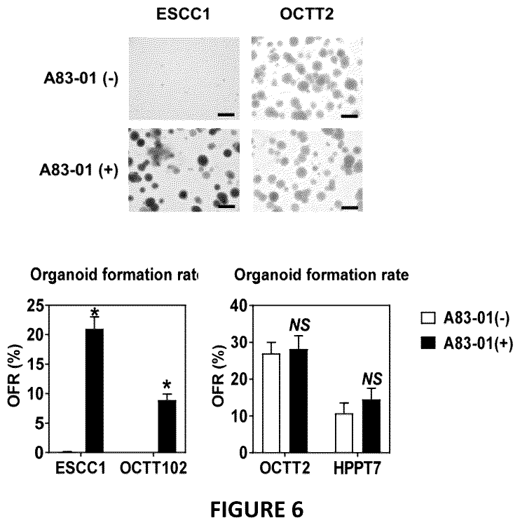

FIG. 6 shows that inhibition of TGF-receptor signaling permits PDO formation by a subset of SCC tumor samples.

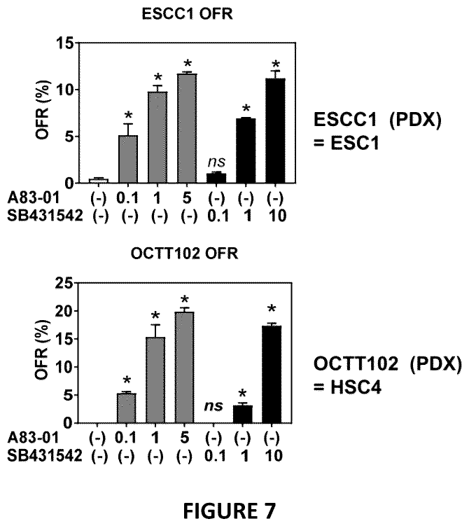

FIG. 7 shows the dose-dependent effects of TGF-β receptor signaling inhibitors upon organoid formation by ESCC1 and OCTT102 cells.

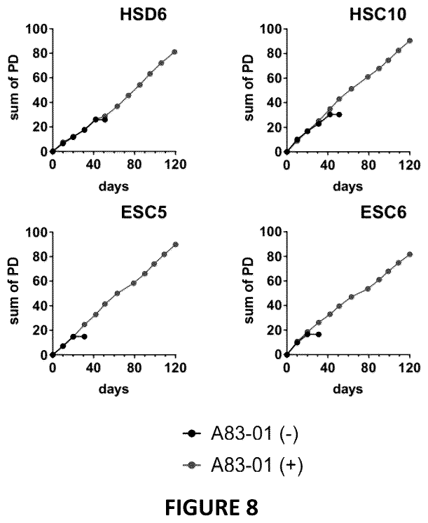

FIG. 8 shows that inhibition of TGF-β receptor signaling may extend replicative lifespan in a subset of neoplastic PODs

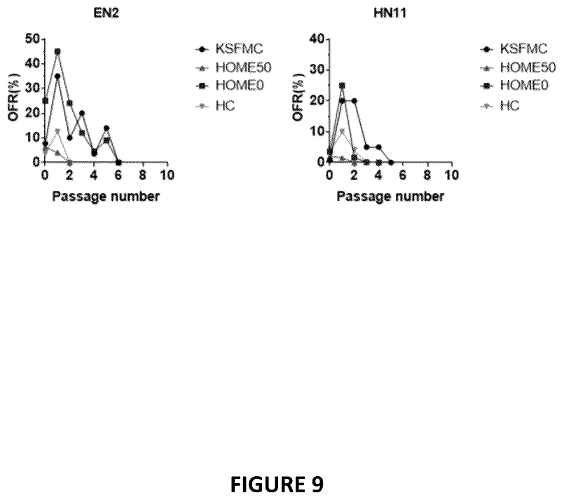

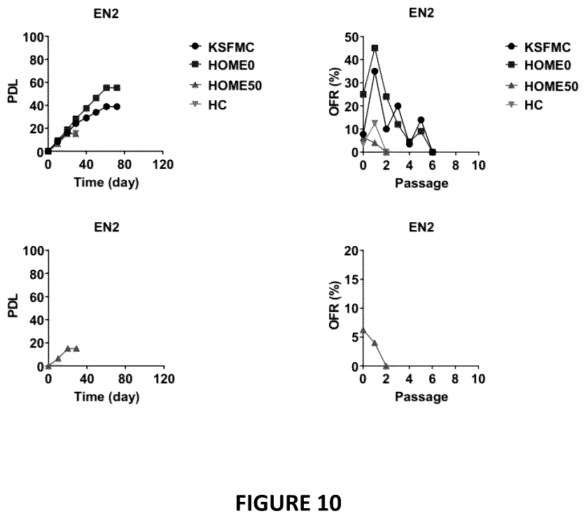

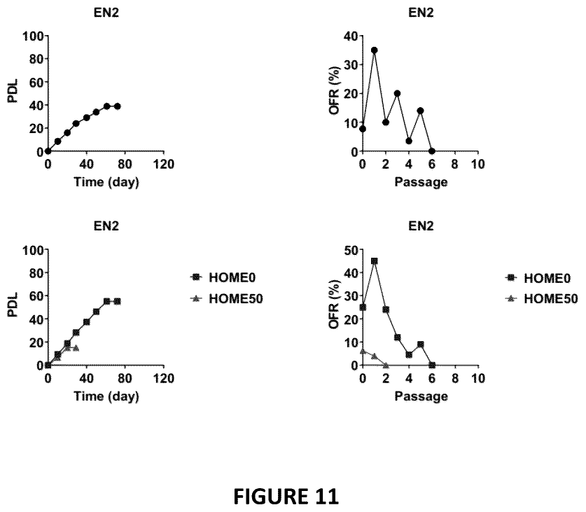

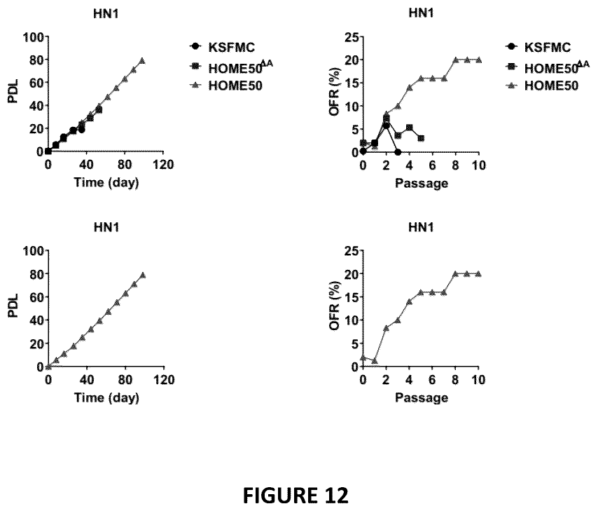

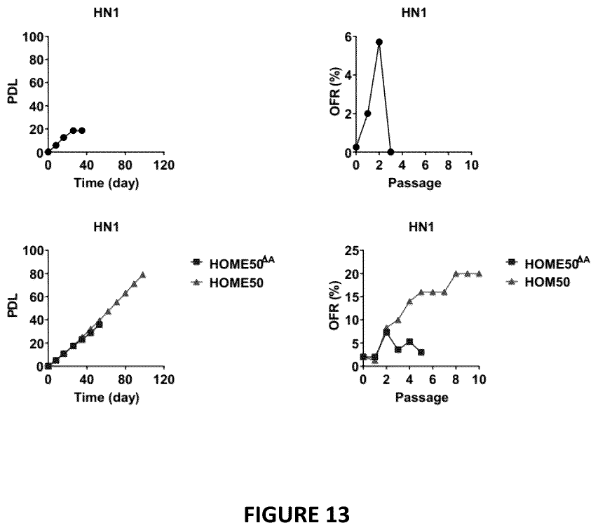

FIGS. 9-13 show organoid formation rate and population doubling (PDL) measured at various time points for organoids grown in different media (KSFMC, HOME0, HOME50, or HC). Organoids EN2, HN1*, HN11 were grown from normal mucosa (EN, esophageal normal; HN, head-and-neck normal). HSC5 and HSC6 were from head and neck squamous cell cancer tumors. HSD5 and HSD6 were from head and neck/oral preneoplasia/dysplasia.

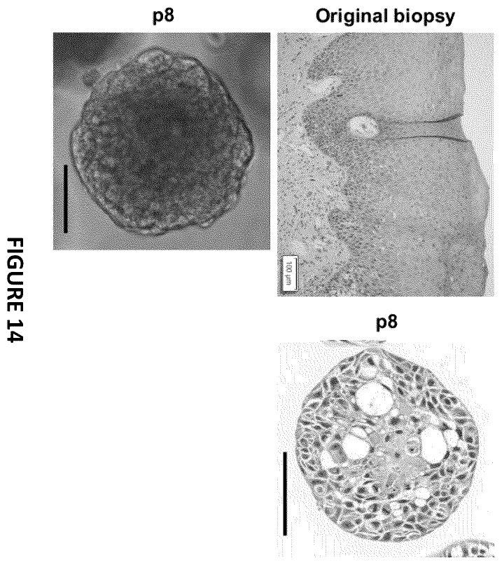

FIG. 14 shows that PDO was established from a 78 y.o. male with oral (tongue) squamous dysplasia. Established organoids were grown indefinitely (>10 passages) in HOME50 medium, displaying moderate atypia (grade 2). Scale bar, 100 μm.

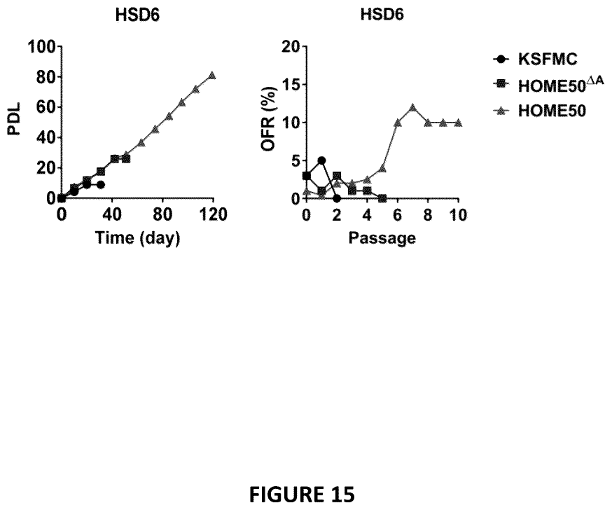

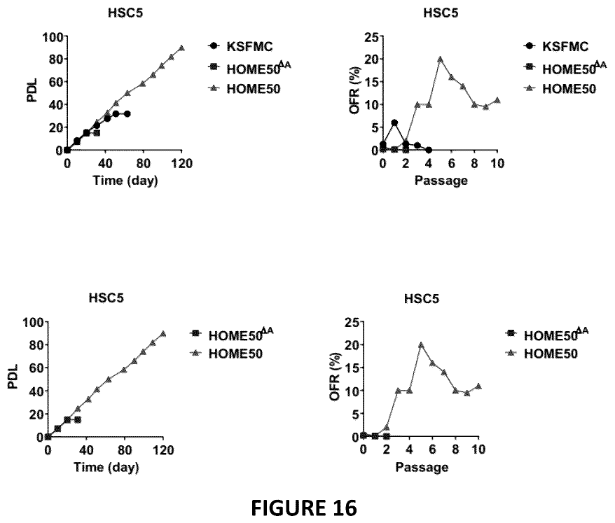

FIGS. 15 and 16 show organoid formation rate (OFR) and population doubling (PDL) measured at various time points for organoids grown in different media (KSFMC, HOME0, HOME50, or HC). Organoids EN2, HN1*, HN11 were grown from normal mucosa (EN, esophageal normal; HN, head-and-neck normal). HSC5 and HSC6 were from head and neck squamous cell cancer tumors. HSD5 and HSD6 were from head and neck/oral preneoplasia/dysplasia.

### Example 14

Co-Culturing PDO with Cancer Associate Fibroblasts (CAF)

Cancer associated fibroblasts (CAFs) are critical components of the tumor cell microenvironment, and promoter tumor cell migration and invasion. CAFs are activated through autocrine signals and foster tumor cell progress through paracrine signals.

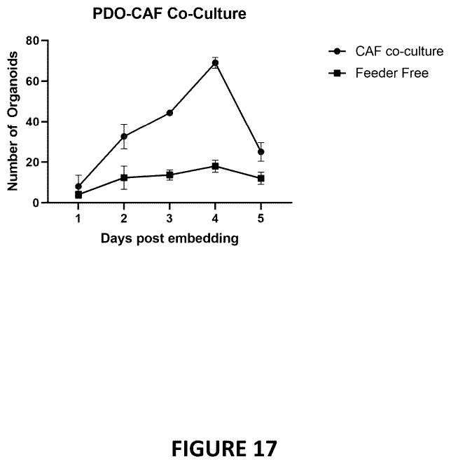

As shown in FIG. 17, an exemplary human CAF feeder cell line was shown to accelerate 3D patient derived organoid growth (specifically, esophageal adenocarcinomas) in terms of size and number. After 48 hours there is a clear difference in the number of organoids after two days, which becomes even more pronounced by day four and is still significant at day five.

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

What is claimed is:

1. An organoid medium for producing an esophageal organoid, comprising:
(a) a solubilized membrane matrix;
(b) about 1 μM to about 20 μM of a TGF-B inhibitor; and
(c) less than about 50 ng/ml of epidermal growth factor (EGF).

2. The medium of claim 1, further comprising from about 0.5×to about 1.5×N-2 supplement.

3. The medium of claim 2, further comprising advanced DMEM+/+/+, 1×B27 supplement, 1.25 mmol/L N-acetyl-L-cysteine, 4% R-spondin, and 4% Noggin.

4. The medium of claim 1, further comprising:
(d) a feeder layer comprising a plurality of cancer associated fibroblasts or fetal esophageal fibroblasts.

5. The medium of claim 3, further comprising:
(d) a feeder layer comprising a plurality of cancer associated fibroblasts or fetal esophageal fibroblasts.

6. A method for producing an esophageal organoid, comprising:
(a) isolating cells from an esophageal biopsy to provide isolated cells;
(b) culturing the isolated cells in a three-dimensional culture comprising an organoid medium for a time sufficient to produce at least one organoid, wherein the organoid medium comprises
(i) a solubilized membrane matrix;
(ii) about 1 μM to about 20 μM of a TGF-B inhibitor; and
(iii) less than about 50 ng/ml of epidermal growth factor (EGF).

7. The method of claim 6, wherein the organoid medium further comprises from about 0.5×to about 1.5×N-2 supplement.

8. The method of claim 7, wherein the organoid medium further comprises advanced DMEM+/+/+, 1×B27 supplement, 1.25 mmol/L N-acetyl-L-cysteine, 4% R-spondin, and 4% Noggin.

9. The method of claim 6, wherein the organoid medium further comprises a feeder layer comprising a plurality of cancer associated fibroblasts or fetal esophageal fibroblasts.

10. The method of claim 8, wherein the organoid medium further comprises a feeder layer comprising a plurality of cancer associated fibroblasts or fetal esophageal fibroblasts.

11. The method of claim 6, wherein the cells are cells from an esophageal adenocarcinoma (EAC) or esophageal squamous cell carcinoma (ESCC).

12. An esophageal organoid produced by the method of claim 6.

13. An esophageal organoid produced by the method of claim 7.

14. An esophageal organoid produced by the method of claim 8.

15. An esophageal organoid produced by the method of claim 9.

16. An esophageal organoid produced by the method of claim 10.

17. An esophageal organoid produced by the method of claim 6, wherein the cells are cells from an esophageal adenocarcinoma (EAC) or esophageal squamous cell carcinoma (ESCC).

18. The esophageal organoid of claim 17, which is capable of being sub-cultured for at least 10 passages.
